(19) United States
(12) Patent Application Publication
(10) Pub. No.: US 2026/0358240 A1
(43) Pub. Date: Nov. 12, 2026

(54) OPTICAL SENSING PLATFORM FOR SYNOVIAL FLUID ANALYSIS

(71) Applicant: THE BOARD OF TRUSTEES OF THE UNIVERSITY OF ARKANSAS, Little Rock, AR (US)

(72) Inventors: Narasimhan RAJARAM, Bentonville, AR (US); Erin DREWKE, Fayetteville, AR (US); Kevin WONG, Little Rock, AR (US); Hanna JENSEN, Fayetteville, AR (US)

(21) Appl. No.: 19/130,545

(22) PCT Filed: Nov. 16, 2023
(86) PCT No.: PCT/US2023/080092

§ 371 Date: May 15, 2025

Related U.S. Application Data
(60) Provisional application No. 63/425,859, filed on Nov. 16, 2022.

(51) Int. Cl. G01N 21/31; G01N 33/49

(52) U.S. Cl. CPC G01N 21/31; G01N 33/49

(57) ABSTRACT

A non-contact sample holder for use with an optical detection system provides for the insertion of sample collection vessels into a housing to block out ambient light, and for optical inspection using an optical detection system without contamination of the fluid sample. Advantageously, this enables rapid fluid sample testing (e.g., at the patient bed side). Non-contact analysis of a fluid sample can be performed, such that the integrity of the sample is not lost during testing. This allows for subsequently testing of the fluid sample, if necessary, without loss of validity.

### BACKGROUND

Septic arthritis, a bacterial infection of a joint such as a knee, hip, or shoulder, is a medical emergency that has substantial mortality and morbidity. A patient usually presents with a hot, swollen joint, and unless adequate treatment, such as draining the joint and antibiotic treatment, is administered promptly, the condition can lead to irreversible joint destruction or a systemic septic infection potentially leading to death. Septic arthritis is typically diagnosed by drawing a small volume of joint fluid into a syringe and analyzing the sample for the presence of white blood cells which appear when an infection is present. This laboratory test can typically take about 30 minutes to over an hour to complete but is essential in determining the course of clinical care. Usually, a threshold of 50,000 white blood cells (WBC) is considered a reason to require immediate operative treatment. In the case of pediatric patients, who commonly must be sedated for the drawing of the joint fluid, this means that they wait for the laboratory result under anesthesia. For adults, the waiting period adds critical time as an operative team is usually not assembled before the results are complete. To reduce the time interval that pediatric patients are under anesthesia, and to expedite this crucial laboratory test for all patients, there is a need for rapid diagnostic testing that can allow physicians to quickly ascertain whether a joint is infected and determine the overall level of infection so the joint can be treated appropriately in a timely manner.

### SUMMARY OF THE DISCLOSURE

The present disclosure addresses the aforementioned drawbacks by providing a non-contact optical detection method for analyzing a biological fluid sample. The method includes acquiring a biological fluid sample from a subject in a sample collection vessel. Optical data are acquired by inspecting the biological fluid sample using an optical detection system without contacting the biological fluid sample with the optical detection system. The optical data are analyzed with a computer system, and a presence of a pathological condition is determined based on analyzing the optical data with the computer system.

It is another aspect of the present disclosure to provide a non-contact optical detection sample holder device, which includes a housing extending from a proximal end to a distal end. The housing includes a first opening formed in the proximal end of the housing and configured to receive a sample collection vessel containing a biological fluid sample of a subject in an ambient light-tight manner; a channel partially extending within the housing from the first opening towards the distal end of the housing, the channel being configured to hold the sample collection vessel within the housing; and a second opening extending from an outer surface of the housing to an inner surface of the channel and configured to receive an optical probe for directing light into and receiving light reflected back from the channel.

It is yet another aspect of the present disclosure to provide a non-contact optical analysis system that includes a light source, an optical detection device, an optical probe in optical communication with the light source and the optical detection device, and a sample holder that includes a housing extending from a proximal end to a distal end. The housing further includes a first opening formed in the proximal end of the housing and configured to receive a sample collection vessel containing a biological fluid sample of a subject in an ambient light-tight manner; a channel partially extending within the housing from the first opening towards the distal end of the housing, the channel being configured to hold the sample collection vessel within the housing; and a second opening extending from an outer surface of the housing to an inner surface of the channel and configured to receive the optical probe. The optical probe is configured to receive and direct light from the light source towards the channel, and to receive and direct reflected light from the channel to the optical detection device.

The foregoing and other aspects and advantages of the present disclosure will appear from the following description. In the description, reference is made to the accompanying drawings that form a part hereof, and in which there is shown by way of illustration one or more embodiments. These embodiments do not necessarily represent the full scope of the invention, however, and reference is therefore made to the claims and herein for interpreting the scope of the invention.

### BRIEF DESCRIPTION OF THE DRAWINGS

FIG. 1A shows an example sample holder according to aspects of the present disclosure.

FIG. 1B shows an example sample holder and tray according to aspects of the present disclosure.

FIG. 2A shows an optical system using the sample holder of FIG. 1A according to aspects of the present disclosure.

FIG. 2B shows an optical system using the sample holder and tray of FIG. 1B according to aspects of the present disclosure.

FIG. 3 shows the light transmission and reflection for optical detection according to aspects of the present disclosure.

FIG. 4 shows the light transmission and reflection within a biological fluid sample according to aspects of the present disclosure.

FIG. 5 shows optical spectra of different fluid samples interrogated by the optical system according to aspects of the present disclosure.

FIGS. 6A and 6B illustrate cross-sectional views of example sample holder devices without an integrated optical detection device (FIG. 6A) and with an integrated or removably coupled optical detection device (FIG. 6B).

FIG. 7 is a flow chart presenting the steps of an example method for performing non-contact optical detection of a fluid sample according to aspects of the present disclosure.

FIG. 8A is a schematic of a laser emitting diode (LED) light source illuminating a sample in a collection vessel (front view).

FIG. 8B is a schematic of an LED light source illuminating a sample in a collection vessel (side view).

FIG. 9A is a plot of the diffuse reflectance spectrum (DRS) results for the dye-containing phantoms at four different scattering levels.

FIG. 9B is a plot of the DRS results as a sparse matrix.

FIG. 9C is a plot of the DRS results as a sparse matrix and the resulting lookup table (LUT).

FIG. 10A is a plot of the DRS (scattering=1.01 mm⁻¹ and [Hb]=1.0 mg/mL, Chi-square=0.21) and the LUT fit from the validation set.

FIG. 10B is a scatterplot of know vs. measured values of scattering the entire validation set. The solid line indicates an ideal fit.

FIG. 10C is a scatterplot of known versus measured values of absorption for the entire validation set. The solid line indicates an ideal fit.

FIG. 11 is a block diagram of an example non-contact biological fluid analysis system in accordance with some embodiments described in the present disclosure.

FIG. 12 is a block diagram of example components that can implement the non-contact biological fluid analysis system of FIG. 11.

### DETAILED DESCRIPTION

Described herein is a non-contact sample holder for use with an optical detection system, and a method for performing optical detection of fluid samples components in a non-contact manner. The sample holder provides for the insertion of sample collection vessels into a housing to block out ambient light and for optical inspection using an optical detection system without contamination of the fluid sample. Advantageously, the systems and methods described in the present disclosure enable rapid fluid sample testing (e.g., at the patient bed side), thereby improving the clinical workflow and reducing the amount of time that a patient is under anesthesia. Furthermore, the systems and methods described in the present disclosure provide for non-contact analysis of a fluid sample, such that the integrity of the sample is not lost during testing. This allows for subsequently testing of the fluid sample, if necessary, without loss of validity.

Many medical and research facilities rely on biological samples for the identification and diagnosis of various constituents and biological anomalies. Most of the pre-existing modalities use invasive machines or excised samples. The systems and methods described in the present disclosure provide for a noncontact method and device for sensing of tissue/fluid biology using optical fibers. This method allows for faster and non-destructive evaluation of biological samples. Most measurements involving optical spectroscopy (the technique) are performed in contact with tissue or fluids. The disclosed systems and methods allow for such measurements in a non-contact manner with biological samples or fluids placed in a tube using a 3D printed tube holder.

As will be described below in more detail, the optical fibers of an optical probe can be arranged in a circular pattern for the detection of reflected light surrounding an optical fiber that emits light. This optical probe is then inserted into a customized light blocking sheath where the sample of interest is placed. The light-blocking sheath houses sample tubes of various sizes and the optical probe sits flush on top of the tube. The emitted light passes through the sample tube and different light wavelengths are either scattered back to the detecting fibers or absorbed by the sample. The light collected in the detecting fibers is fed into a spectrometer and analyzed using computer software. The software used first prompts the user for the sample identification input and then tells the user to insert the sample. Once the sample is inserted into the light blocking sheath, the probe is placed on top of the sample tube inside the light blocking sheath. The user then notifies the system that the sample is inserted and the software starts acquiring the data. Once collected the software continues to use the data and fit to look up tables and give the user an end determination of the desired sample constituents. The software processing takes less than a minute to complete.

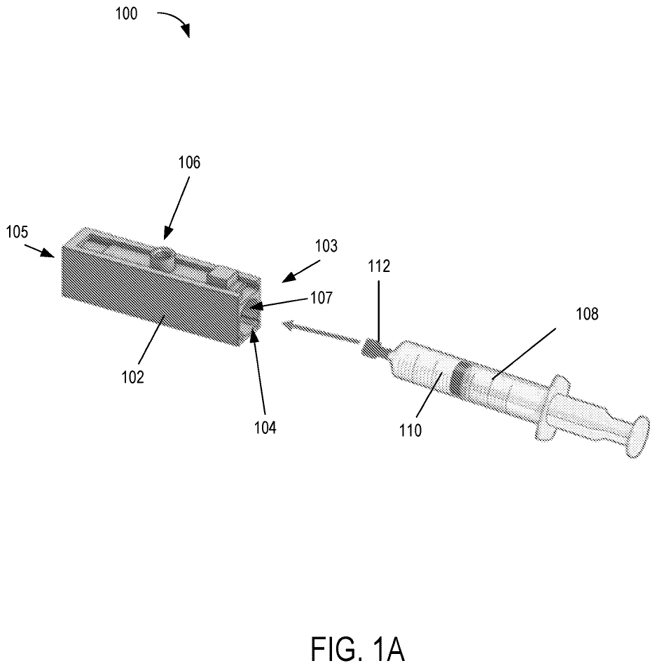

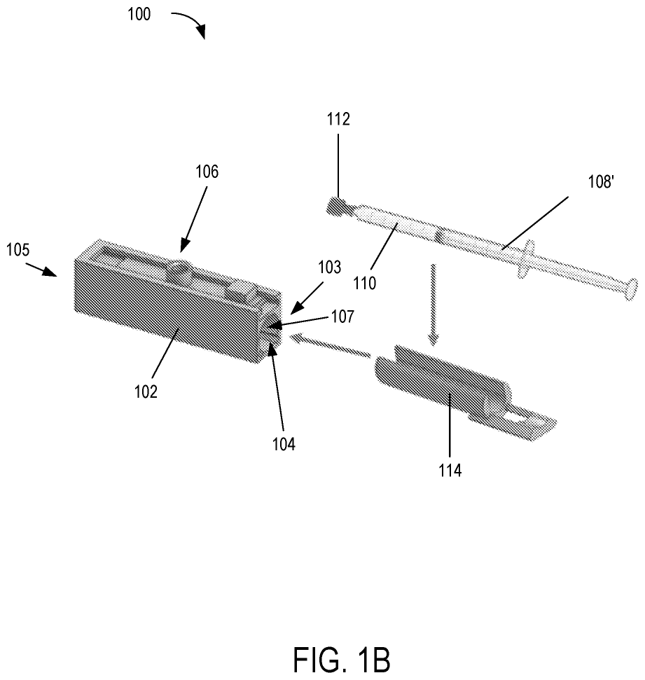

As show in FIG. 1A and FIG. 1B, a non-contact optical detection sample holder device 100 according to some aspects includes a housing 102 extending from a proximal end 103 to a distal end 105. A first opening 104 is formed in the proximal end 103 of the housing 102 and partially extends as a channel 107 toward the distal end 105. The housing 102 further includes a second opening 106 that extends from an outer surface of the housing 102 to an inner surface of the channel (not shown) for receiving an optical probe. A sample collection vessel 108 (e.g., a syringe, test tube, vial) containing biological fluid sample 110 is inserted into the opening 104 of the housing 102. The biological fluid sample 110 may be synovial fluid from the joint of a subject. The tip 112 of the sample collection vessel 108 contacts the distal end of the channel (not shown) within the housing 102. The distal end of the channel may include a recessed portion (not shown) to receive and securely hold a tip 112 of the sample collection vessel 108. For example, the recessed portion may be sized or otherwise dimensioned to securely receive the tip 112 of the sample collection vessel 108. Additionally or alternatively, a flexible baffle, series of bristles, or other means for securely holding the tip 112 of the sample collection vessel 108 in place can also be arranged within the recessed portion. These latter configurations can be advantageous for accommodating sample collection vessels 110 with different sized tips 112 within the same sample holder device 100.

FIG. 1B includes a tray 114 for holding a sample collection vessel 108′ that has a smaller cross-sectional area than the cross-sectional area of the first opening 104, such that the sample collection vessel 108′ is stabilized within the housing 102 in a light-tight manner to prevent ambient light from entering the channel of the housing 102. Advantageously, one or more trays 114 can be provided with the sample holder device 100 to allow for differently sized sample collection vessels 108, 108′ within the same sample holder device 100.

The housing 102 and the tray(s) 114 may be manufactured using various techniques. As a non-limiting example, the housing 102 and the tray(s) 114 may be manufactured using an additive manufacturing process, such as 3D printing.

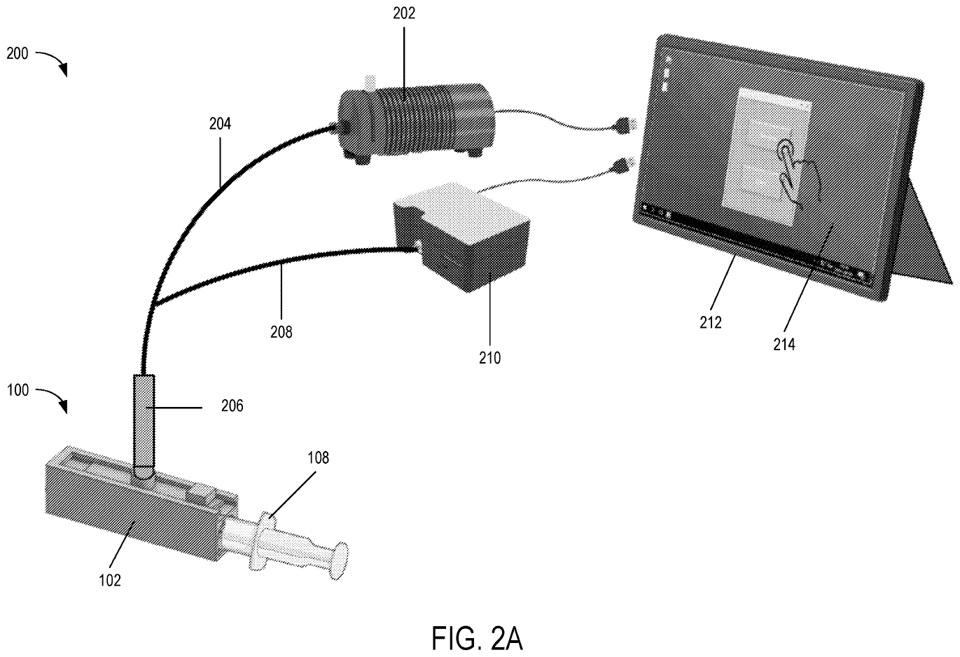

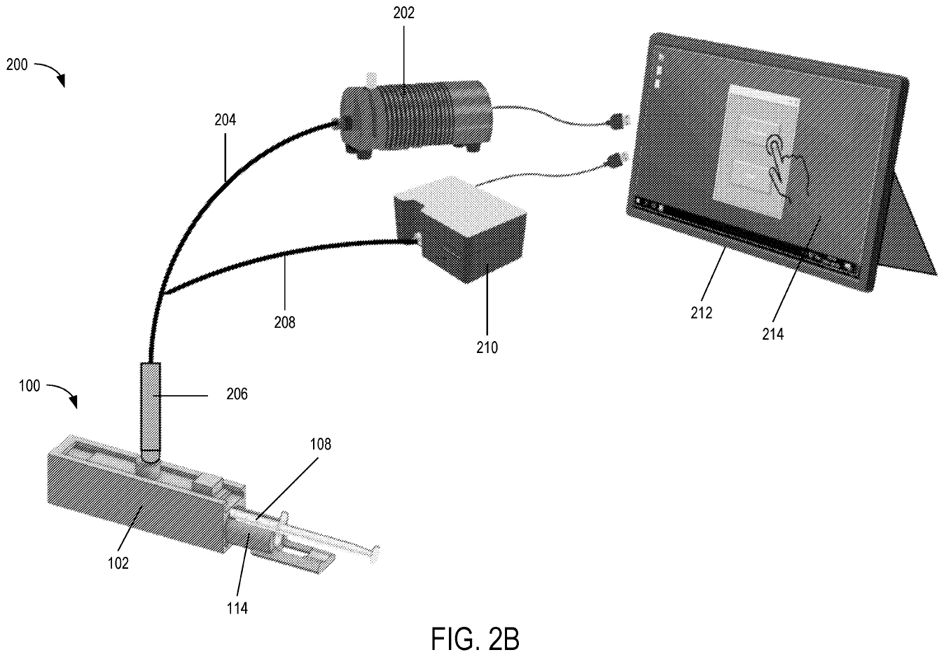

FIG. 2A and FIG. 2B illustrate the non-contact optical detection sample holder devices 100 of FIGS. 1A and 1B in a non-contact optical detection system 200. A light source 202 generates and transmits light through a first cable 204 (e.g., a first fiber optic cable) to optical probe 206. The light source 202 may be a broadband light source, such as a broadband white light source. For instance, the light source 202 may be a halogen lamp. Alternatively, the light source 202 may be a light source that emits light over a narrower range of wavelengths. As a non-limiting example, the light source may emit light over a range of 450-650 nm. For instance, the light source 202 may be one or more pairs of light emitting diodes (LEDs), a laser light source, or so on. The optical probe 206 may be inserted into the second opening 106 of the housing 102 to direct light into the channel of the housing 102 and receive reflected light. The reflected light is received by the optical probe 206 and transmitted through a second cable 208 (e.g., a second fiber optic cable) to an optical detection system 210 for analysis of the optical data. The optical detection system 210 may be, for example, a spectrometer. The first cable 204 and second cable 208 may be fiber optic cables. The optical data may be output to a display 212 with screen 214. The screen 214 may be a touch screen for user input.

The optical probe 206 may contact the outer surface of the sample collection vessel 108, 108′. The distal end of the optical probe 206 contacting the sample collection vessel 108, 108′ may have a complementary shape to the outer surface of the sample collection vessel 108, 108′ to maximize surface contact. For example, the optical probe 206 may have a contact surface that is concave to match the convexity of the sample collection vessel 108, 108′.

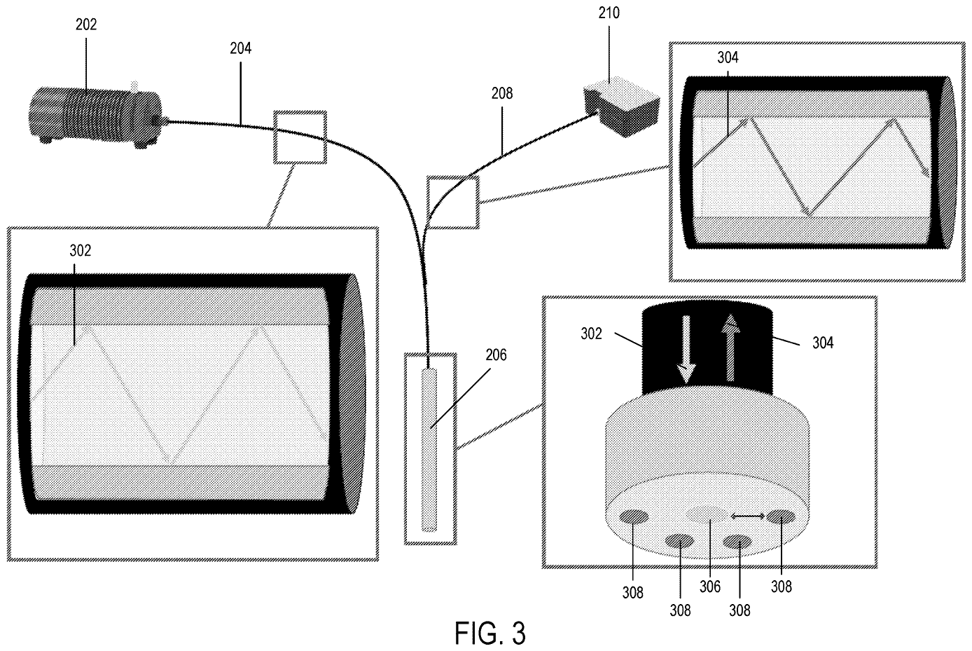

FIG. 3 shows the transmitted light 302 traveling from the light source 202 through the first cable 204 to the probe 206. The transmitted light 302 is transmitted from the probe 206 from one or more probe openings 306 (e.g., openings that are optically coupled to one or more fibers of the first cable 204). Reflected light 304 is received through one or more probe openings 308 (e.g., openings that are optically coupled to one or more fibers of the second cable 208) of probe 206 and travels through the second cable 208 to the optical detection system 210. The distance between the source opening(s) 306 and the detection opening(s) 308 can be referred to as the source-detection distance. The optical probe 206 can be designed with a source-detection distance that is selected to optimize optical detection within the fluid sample. For instance, the source-detection distance can be selected to minimize light receiving light that has traveled through both the near and far walls of the sample collection vessel 108, 108′. As a non-limiting example, the source-detection distance may be selected from a range of 700 μm-2.25 mm.

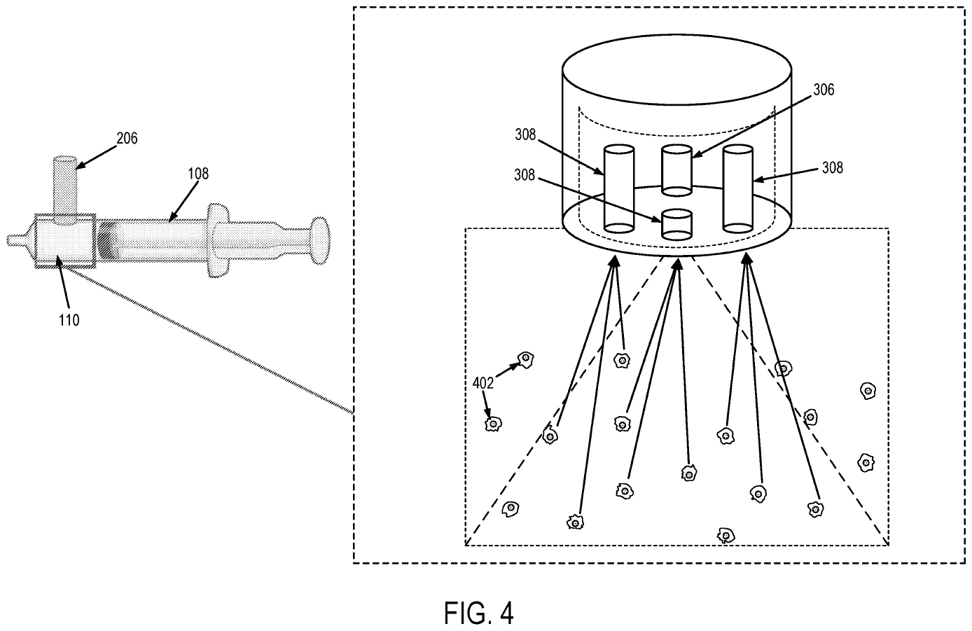

FIG. 4 illustrates the transmission of light via optical probe 206 into the sample collection vessel 108 and fluid sample 110. Transmitted light 302 exits probe opening 306 and irradiates fluid sample 110. Scattering particles 402 within the fluid sample may scatter reflected light 304 through probe openings 308. The fluid sample may be synovial fluid, and the scattering particles 402 may be white blood cells.

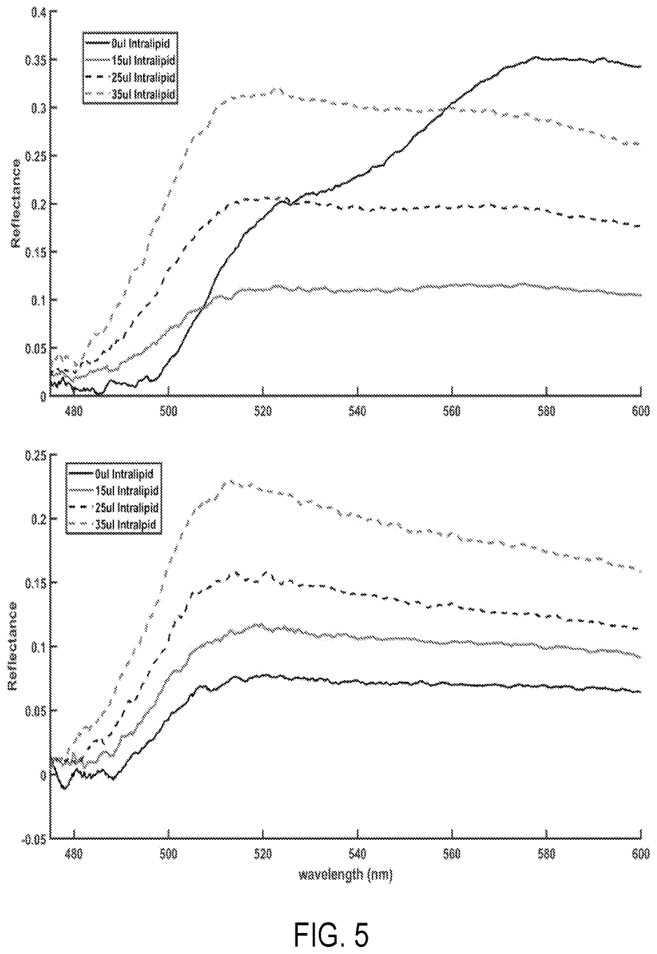

FIG. 5 illustrates example reflectance plots acquired from 10 mL (top) and 1.0 mL (bottom) fluid samples containing different concentrations of scattering particles to simulate different concentrations of white blood cells.

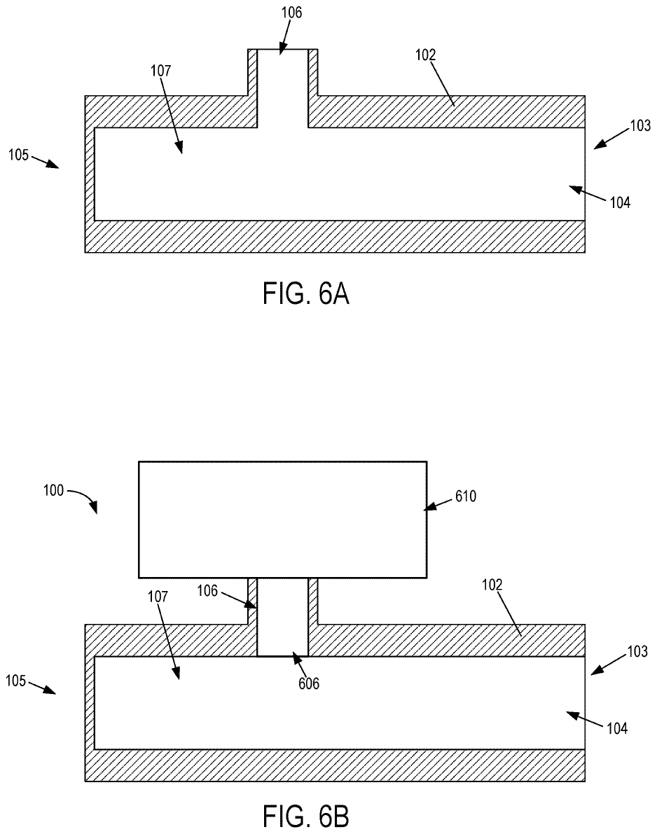

FIGS. 6A and 6B illustrate a cross-sectional view of an example sample holder device 100. The embodiment illustrated in FIG. 6A is similar to the sample holder device 100 shown in FIGS. 1A and 1B, in which an optical probe 206 is introduced into the second opening 106 (e.g., as shown in FIGS. 2A and 2B). As described above, in these embodiments, a separate light source (e.g., light source 202) and optical detection system (e.g., optical detection system 210) are utilized. In the embodiment illustrated in FIG. 6B, a compact optical detection system 610 having an optical probe 606 is coupled to the second opening 106. In these instances, the compact optical detection system 610 can be removably coupled to the sample holder device 100, or the compact optical detection system 610 may be integral with the sample holder device 100 (i.e., attached via a fixed coupling). In either instance, the compact optical detection system 610 can include a light source (e.g., one or more LEDs) and the relevant detection electronics for processing the optical data received from the fluid sample. Additionally or alternatively, the compact optical detection system 610 can include a wireless communication device that is capable of transmitting the acquired optical data to a separate computer system for processing.

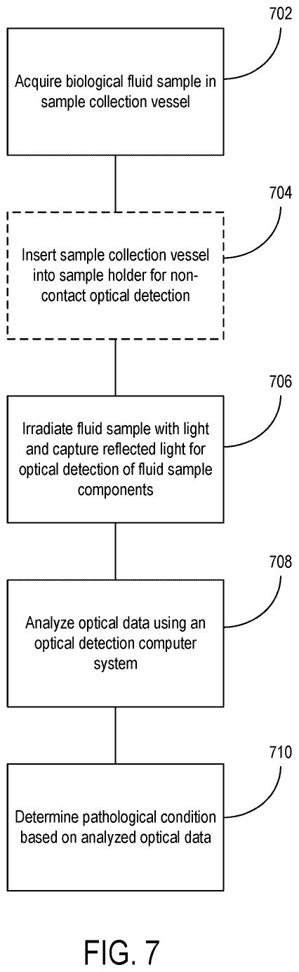

Referring to FIG. 7, a flowchart is illustrated as presenting the steps of an example method for non-contact optical detection for analyzing biological fluid samples. The method includes acquiring a biological fluid sample from a subject, as indicated at step 702. For example, the biological fluid sample may be a synovial fluid sample extracted from a joint of the subject. In a non-limiting example, the biological fluid sample is contained in a sample collection vessel (e.g., a syringe), which may be inserted into sample holder for non-contact optical detection, as indicated at step 704. Once inserted into the sample holder, light from an optical probe irradiates the biological fluid sample and reflected light scattered from sample components acting as scattering particles within the fluid sample is received by the optical probe for optical detection, as indicated at step 706.

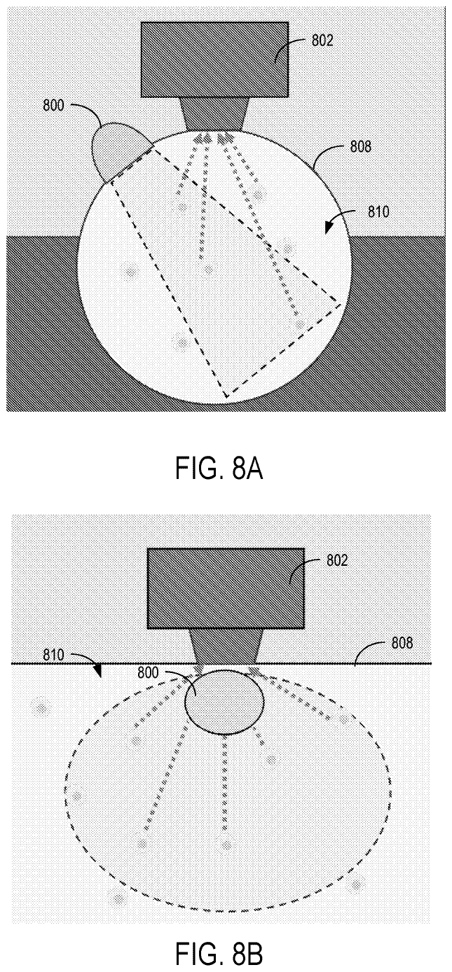

Alternatively, the biological fluid sample may be irradiated without an optical probe, such as by directly irradiating the biological fluid sample using a light source that transmits light through open air, or the like. Additionally or alternatively, the reflected light scattered from the sample components may be received by an optical detection device other than the optical probe. For instance, the received light may be detected using a photodetector or other optical detection device capable of detecting light that has been transmitted through open air, or the like. As a non-limiting example, as illustrated in FIGS. 8A-8B, the biological fluid sample 800 may be irradiated with a laser emission diode (LED) light source 800, or the like, and the reflected light may be detected by a photodetector 802. In a non-limiting example, the LED light source 800 may contact the syringe 808 (FIG. 8A-8B). A photodetector 802 for collecting the light from the LED light source 800 may also contact the syringe 808.

The received light can be stored as optical data. The sample components may include white blood cells. The optical detection method may be diffuse reflectance spectroscopy (DRS). Received optical data are then analyzed using an optical detection computer system, as indicated at step 708. A computer system further determines a pathological condition based on the analyzed optical data, as indicated at step 710. The pathological condition may be a bacterial infection or septic arthritis. As an example, the pathological condition may be determined based on analyzing the optical data to estimate a number of white blood cells in the fluid sample. The measured reflectance values stored in the optical data can indicate the presence of scatterers, such as white blood cells. By comparing the reflectance values to reference data, an estimate of the number of white blood cells can be determined. Then, based on the estimate of the number of white blood cells, the pathological condition can be determined. For instance, if the analysis of the optical data indicates that the white blood cell count in the fluid sample is at or above 50,000, then the fluid sample can be labeled as corresponding to a pathological condition of inflammation, such as bacterial infection of septic arthritis.

In a non-limiting example, determining a presence of a pathological condition includes comparing the optical data to a threshold value. For example, white blood cells can be characterized based on their light scattering properties. To determine whether the WBC in a fluid sample is at or under a threshold value, scattering levels in light interaction models should be accurately measured. In a non-limiting example, the optical data is compared to a non-contact lookup table (LUT) to accurately measure light-tissue interactions through a barrier developed for optical probes, where the light barrier is axially offset from tissue.

In a non-limiting example, the LUT is built by using phantoms of known optical properties in syringes to accurately quantify scattering and absorption properties. For example, the LUT is made by measuring the reflectance of liquid phantoms containing polymer microspheres, water, and dye. In a non-limiting example, the polymer microspheres include polystyrene microspheres. Further, the microspheres may have a diameter of 0.75 um. Polystyrene bead may be used to simulate scattering and the dye may be used to show a range of absorption values. In a non-limiting example, the scattering coefficient μ′ₛ may be calculated using Mei theory and the absorption coefficient may be measured using a spectrophotometer. In this example set-up, a matrix was created using eight total phantoms with four levels of scattering only and four with the same levels of scattering with dye added.

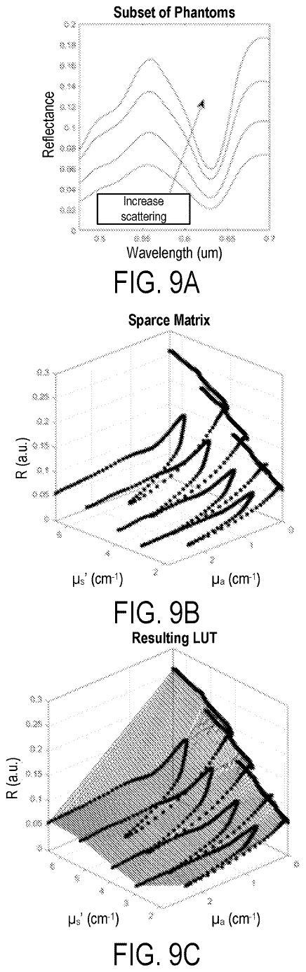

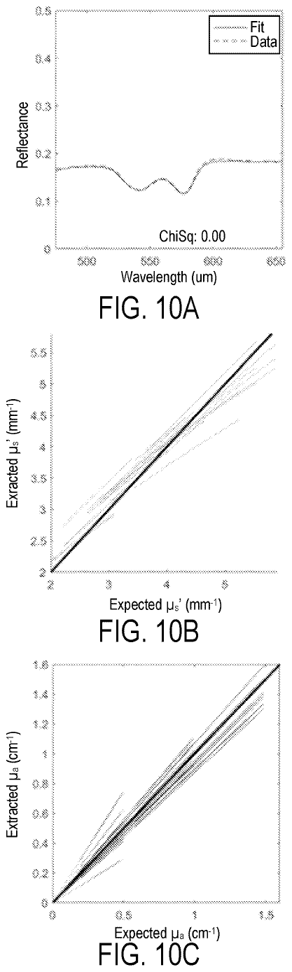

In this example, the LUT is validated by creating 24 phantoms having eight scattering levels and three hemoglobin concentrations (0.5, 1, and 1.5 mg/mL) for the absorbers. The non-contact LUT performance for 1 ml syringe samples was compared to a traditional contact LUT, resulting in approximately 6% errors for scattering and 12% for absorption values which is consistent with a contact LUT. FIGS. 9A-9C and FIGS. 10A-10C show the results of the non-contact LUT validation.

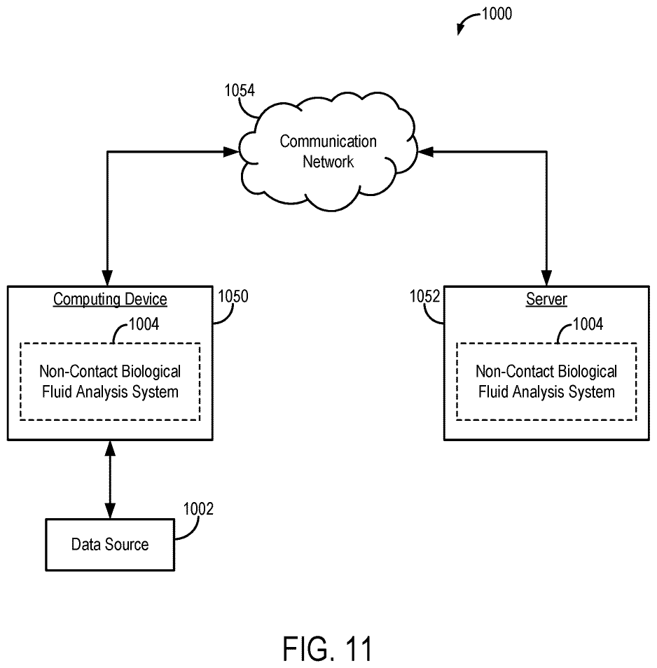

Referring now to FIG. 11, an example of a system 1000 for non-contact biological fluid analysis in accordance with some embodiments of the systems and methods described in the present disclosure is shown. As shown in FIG. 11, a computing device 1050 can receive one or more types of data (e.g., optical data) from data source 1002. In some embodiments, computing device 1050 can execute at least a portion of a non-contact biological fluid analysis system 1004 to determine the presence, or likelihood of the presence, of a pathological condition in a subject from data received from the data source 1002.

Additionally or alternatively, in some embodiments, the computing device 1050 can communicate information about data received from the data source 1002 to a server 1052 over a communication network 1054, which can execute at least a portion of the non-contact biological fluid analysis system 1004. In such embodiments, the server 1052 can return information to the computing device 1050 (and/or any other suitable computing device) indicative of an output of the non-contact biological fluid analysis system 1004.

In some embodiments, computing device 1050 and/or server 1052 can be any suitable computing device or combination of devices, such as a desktop computer, a laptop computer, a smartphone, a tablet computer, a wearable computer, a server computer, a virtual machine being executed by a physical computing device, and so on. The computing device 1050 and/or server 1052 can also reconstruct images from the data.

In some embodiments, data source 1002 can be any suitable source of data (e.g., optical data, processed optical data), such as a spectrometer or other optical detection system, another computing device (e.g., a server storing optical data, processed optical data), and so on. In some embodiments, data source 1002 can be local to computing device 1050. For example, data source 1002 can be incorporated with computing device 1050 (e.g., computing device 1050 can be configured as part of a device for measuring, recording, estimating, acquiring, or otherwise collecting or storing data). As another example, data source 1002 can be connected to computing device 1050 by a cable, a direct wireless link, and so on. Additionally or alternatively, in some embodiments, data source 1002 can be located locally and/or remotely from computing device 1050, and can communicate data to computing device 1050 (and/or server 1052) via a communication network (e.g., communication network 1054).

In some embodiments, communication network 1054 can be any suitable communication network or combination of communication networks. For example, communication network 1054 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 1054 can be a local area network, a wide area network, a public network (e.g., the Internet), a private or semi-private network (e.g., a corporate or university intranet), any other suitable type of network, or any suitable combination of networks. Communications links shown in FIG. 11 can each be any suitable communications link or combination of communications links, such as wired links, fiber optic links, Wi-Fi links, Bluetooth links, cellular links, and so on.

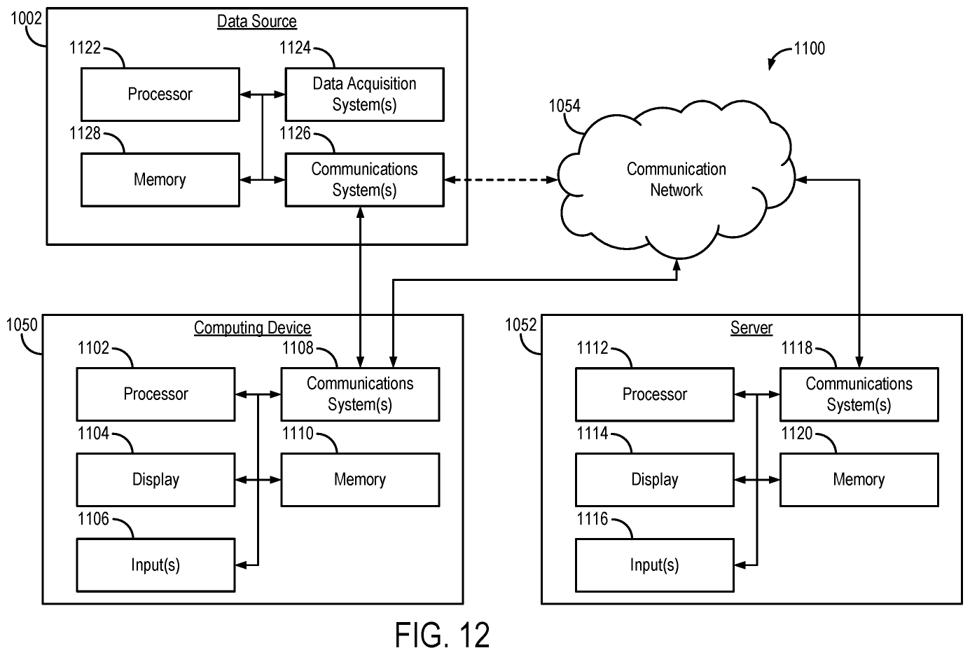

Referring now to FIG. 12, an example of hardware 1100 that can be used to implement data source 1002, computing device 1050, and server 1052 in accordance with some embodiments of the systems and methods described in the present disclosure is shown.

As shown in FIG. 12, in some embodiments, computing device 1050 can include a processor 1102, a display 1104, one or more inputs 1106, one or more communication systems 1108, and/or memory 1110. In some embodiments, processor 1102 can be any suitable hardware processor or combination of processors, such as a central processing unit (“CPU”), a graphics processing unit (“GPU”), and so on. In some embodiments, display 1104 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some embodiments, inputs 1106 can include any suitable input devices and/or sensors that can be used to receive user input, such as a keyboard, a mouse, a touchscreen, a microphone, and so on.

In some embodiments, communications systems 1108 can include any suitable hardware, firmware, and/or software for communicating information over communication network 1054 and/or any other suitable communication networks. For example, communications systems 1108 can include one or more transceivers, one or more communication chips and/or chip sets, and so on. In a more particular example, communications systems 1108 can include hardware, firmware, and/or software that can be used to establish a Wi-Fi connection, a Bluetooth connection, a cellular connection, an Ethernet connection, and so on.

In some embodiments, memory 1110 can include any suitable storage device or devices that can be used to store instructions, values, data, or the like, that can be used, for example, by processor 1102 to present content using display 1104, to communicate with server 1052 via communications system(s) 1108, and so on. Memory 1110 can include any suitable volatile memory, non-volatile memory, storage, or any suitable combination thereof. For example, memory 1110 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some embodiments, memory 1110 can have encoded thereon, or otherwise stored therein, a computer program for controlling operation of computing device 1050. In such embodiments, processor 1102 can execute at least a portion of the computer program to present content (e.g., images, user interfaces, graphics, tables), receive content from server 1052, transmit information to server 1052, and so on. For example, the processor 1102 and the memory 1110 can be configured to perform the methods described herein (e.g., the data analysis steps of the method of FIG. 7).

In some embodiments, server 1052 can include a processor 1112, a display 1114, one or more inputs 1116, one or more communications systems 1118, and/or memory 1120. In some embodiments, processor 1112 can be any suitable hardware processor or combination of processors, such as a CPU, a GPU, and so on. In some embodiments, display 1114 can include any suitable display devices, such as an LCD screen, LED display, OLED display, electrophoretic display, a computer monitor, a touchscreen, a television, and so on. In some embodiments, inputs 1116 can include any suitable input devices and/or sensors that can be used to receive user input, such as a keyboard, a mouse, a touchscreen, a microphone, and so on.

In some embodiments, communications systems 1118 can include any suitable hardware, firmware, and/or software for communicating information over communication network 1054 and/or any other suitable communication networks. For example, communications systems 1118 can include one or more transceivers, one or more communication chips and/or chip sets, and so on. In a more particular example, communications systems 1118 can include hardware, firmware, and/or software that can be used to establish a Wi-Fi connection, a Bluetooth connection, a cellular connection, an Ethernet connection, and so on.

In some embodiments, memory 1120 can include any suitable storage device or devices that can be used to store instructions, values, data, or the like, that can be used, for example, by processor 1112 to present content using display 1114, to communicate with one or more computing devices 1050, and so on. Memory 1120 can include any suitable volatile memory, non-volatile memory, storage, or any suitable combination thereof. For example, memory 1120 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1120 can have encoded thereon a server program for controlling operation of server 1052. In such embodiments, processor 1112 can execute at least a portion of the server program to transmit information and/or content (e.g., data, images, a user interface) to one or more computing devices 1050, receive information and/or content from one or more computing devices 1050, receive instructions from one or more devices (e.g., a personal computer, a laptop computer, a tablet computer, a smartphone), and so on.

In some embodiments, the server 1052 is configured to perform the methods described in the present disclosure. For example, the processor 1112 and memory 1120 can be configured to perform the methods described herein (e.g., the data analysis steps of the method of FIG. 7).

In some embodiments, data source 1002 can include a processor 1122, one or more data acquisition systems 1124, one or more communications systems 1126, and/or memory 1128. In some embodiments, processor 1122 can be any suitable hardware processor or combination of processors, such as a CPU, a GPU, and so on. In some embodiments, the one or more data acquisition systems 1124 are generally configured to acquire data, images, or both, and can include a spectrometer or other optical detection system (e.g., optical detection system 201, compact optical detection system 610). Additionally or alternatively, in some embodiments, the one or more data acquisition systems 1124 can include any suitable hardware, firmware, and/or software for coupling to and/or controlling operations of an optical detection system. In some embodiments, one or more portions of the data acquisition system(s) 1124 can be removable and/or replaceable.

Note that, although not shown, data source 1002 can include any suitable inputs and/or outputs. For example, data source 1002 can include input devices and/or sensors that can be used to receive user input, such as a keyboard, a mouse, a touchscreen, a microphone, a trackpad, a trackball, and so on. As another example, data source 1002 can include any suitable display devices, such as an LCD screen, an LED display, an OLED display, an electrophoretic display, a computer monitor, a touchscreen, a television, etc., one or more speakers, and so on.

In some embodiments, communications systems 1126 can include any suitable hardware, firmware, and/or software for communicating information to computing device 1050 (and, in some embodiments, over communication network 1054 and/or any other suitable communication networks). For example, communications systems 1126 can include one or more transceivers, one or more communication chips and/or chip sets, and so on. In a more particular example, communications systems 1126 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 1128 can include any suitable storage device or devices that can be used to store instructions, values, data, or the like, that can be used, for example, by processor 1122 to control the one or more data acquisition systems 1124, and/or receive data from the one or more data acquisition systems 1124; to generate images from data; present content (e.g., data, images, a user interface) using a display; communicate with one or more computing devices 1050; and so on. Memory 1128 can include any suitable volatile memory, non-volatile memory, storage, or any suitable combination thereof. For example, memory 1128 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1128 can have encoded thereon, or otherwise stored therein, a program for controlling operation of data source 1002. In such embodiments, processor 1122 can execute at least a portion of the program to generate images, transmit information and/or content (e.g., data, images, a user interface) to one or more computing devices 1050, receive information and/or content from one or more computing devices 1050, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the invention.

1. A non-contact optical detection method for analyzing a biological fluid sample, the method comprising:
acquiring a biological fluid sample from a subject in a sample collection vessel;
acquiring optical data by inspecting the biological fluid sample using an optical detection system without contacting the biological fluid sample with the optical detection system;
analyzing the optical data with a computer system; and
determining a presence of a pathological condition based on analyzing the optical data with the computer system.

2. The method of claim 1, wherein the biological fluid sample comprises synovial fluid extracted from a joint of the subject.

3. The method of claim 1, wherein the sample collection vessel is a syringe.

4. The method of claim 1, further comprising inserting the sample collection vessel into a sample holder.

5. The method of claim 4, wherein the sample holder is configured to receive the sample collection vessel in an ambient light-tight manner.

6. The method of claim 1, wherein the optical data are analyzed with the computer system using diffuse reflectance spectroscopy (DRS).

7. The method of claim 6, wherein determining the presence of the pathological condition is based on analyzing a reflectance value obtained using diffuse reflectance spectroscopy.

8. The method of claim 7, wherein the reflectance value is converted to a count of fluid sample components.

9. The method of claim 8, wherein the fluid sample components are white blood cells.

10. The method of claim 1, wherein the pathological condition is at least one of a bacterial infection and septic arthritis.

11. The method of claim 1, wherein determining a presence of a pathological condition includes comparing the optical data to a threshold value.

12. The method of claim 11, wherein the optical data is compared to at least one threshold value contained in a non-contact lookup table (LUT) to measure light-tissue interactions through a barrier.

13. The method of claim 12, further comprising developing the non-contact LUT by measuring reflectance of a plurality of liquid phantoms containing polymer microspheres, water, and dye.

14. The method of claim 13, wherein the polymer microspheres include polystyrene.

15. The method of claim 1, wherein the optical detection system comprises an optical probe for directing light into and receiving light reflected back from the sample collection vessel.

16. The method of claim 15, wherein determining a presence of a pathological condition includes comparing the optical data to a non-contact lookup table (LUT) to measure light-tissue interactions through a barrier that is axially offset from the optical probe.

17. A non-contact optical detection sample holder device comprising:
a housing extending from a proximal end to a distal end, the housing comprising:
a first opening formed in the proximal end of the housing to receive a sample collection vessel containing a biological fluid sample of a subject in an ambient light-tight manner;
a channel partially extending within the housing from the first opening towards the distal end of the housing to hold the sample collection vessel within the housing; and
a second opening extending from an outer surface of the housing to an inner surface of the channel to receive an optical probe for directing light into and receiving light reflected back from the channel.

18. The device of claim 17, further comprising a tray to hold the sample collection vessel, wherein the tray is dimensioned to be inserted into the first opening of the housing such that the sample collection vessel is maintained in the ambient light-tight manner within the channel and held adjacent the second opening.

19. The device of claim 17, wherein the channel is configured at its distal end to removably couple the sample collection vessel within the channel.

20-23. (canceled)

24. A non-contact optical analysis system comprising:
a light source;
an optical detection device;
an optical probe in optical communication with the light source and the optical detection device;
a sample holder comprising a housing extending from a proximal end to a distal end, the housing comprising:
a first opening formed in the proximal end of the housing to receive a sample collection vessel containing a biological fluid sample of a subject in an ambient light-tight manner;
a channel partially extending within the housing from the first opening towards the distal end of the housing to hold the sample collection vessel within the housing; and
a second opening extending from an outer surface of the housing to an inner surface of the channel to receive the optical probe; and
wherein the optical probe receives light from the light source and directs the light from the light source towards the channel, and receives reflected light from the channel and directs the reflected light from the channel to the optical detection device.

25-29. (canceled)
